(19) United States
(12) Patent Application Publication
(10) Pub. No.: US 2021/0338241 A1
(43) Pub. Date: Nov. 4, 2021

(54) DUAL-MOTOR CLIP APPLICATOR

(71) Applicant: Suzhou Intocare Medical Technology Co., Ltd, Suzhou, Jiangsu (CN)

(72) Inventors: Bingchao Zhou, Suzhou, Jiangsu (CN); Hui Zhang, Suzhou, Jiangsu (CN); Yunfeng Du, Suzhou, Jiangsu (CN); Dianchen Liu, Suzhou, Jiangsu (CN)

(21) Appl. No.: 17/273,050

(22) PCT Filed: Nov. 13, 2019
(86) PCT No.: PCT/CN2019/117885

§ 371 Date: Mar. 3, 2021

(30) Foreign Application Priority Data
Nov. 29, 2018 (CN) 201811439560.1

(51) Int. Cl. A61B 17/128; A61B 17/122

(52) U.S. Cl. CPC A61B 17/128; A61B 2017/00398; A61B 17/122

(57) ABSTRACT

A dual-motor clip applicator includes a working head, and a first motor, a second motor, a first driving assembly, a second driving assembly and a clip cartridge assembly that are provided in the working head. The clip cartridge assembly includes a clip cartridge configured to accommodate a ligation clip and a clip jaw provided on a side of the clip cartridge; the working head further includes a firing rod and a clip-pushing rod, the firing rod is connected to the clip jaw to drive the clip jaw to open or close for firing the ligation clip, the first driving assembly is respectively connected to the firing rod and the first motor, the second driving assembly is respectively connected to the clip-pushing rod and the second motor, and the clip-pushing rod is configured to push the ligation clip in the clip cartridge to enter into the clip jaw.

The present application claims priority of the Chinese Patent Application No. 201811439560.1 filed on Nov. 29, 2018, the disclosure of which is incorporated herein by its reference in its entirety as part of the present application.

### TECHNICAL FIELD

The present disclosure relates to a dual-motor clip applicator, and belongs to the field of medical instruments.

### BACKGROUND

In order to fully expose a surgical visual field during a surgical operation, target tissue at an incision needs to be ligated to stop bleeding. Hemostasis technique has become one of main surgical techniques. Surgical operations on any part of human body almost without exception involve bleeding and stopping bleeding. Generally, a ligation clip applied by a clip applicator is used to clamp the target tissue so as to stop bleeding. A conventional clip applicator is usually single-firing and only applies one ligation clip at one time, and another ligation clip needs to be placed into the clip applicator after the one ligation clip clamps the target blood vessel. However, during the surgical operation, a plurality of ligation clips are required so as to stop bleeding; in this case, the conventional clip applicator has to enter and exit a puncture many times for example during an endoscopic surgical operation, which causes the time of the surgical operation to be too long, increases the bleeding amount of the patient, and adversely affects postoperative recovery of the patient. In addition, the conventional clip applicator completes the delivery of the ligation clip and the firing of the ligation clip only by one shaft, and has strong dependent and restrictive relationships between the delivery of the ligation clip and the firing of the ligation clip so that operation errors are easily caused.

### SUMMARY

The present disclosure aims to provide a dual-motor clip applicator to solve the problems that the conventional clip applicator needs to complete the firing of the ligation clip and the delivery of the ligation clip by one shaft and thus has strong dependent and restrictive relationships between the firing of the ligation clip and the delivery of the ligation clip to easily cause operational errors and the like; the dual-motor clip applicator provided by the embodiments of the present disclosure uses two motors, such that the operation is simpler, the freedom degree of the operation is higher, and the error rate is reduced.

In order to achieve the above objects, the technical solutions of the present disclosure are provided as follows. A dual-motor clip applicator comprises a working head, and a first motor, a second motor, a first driving assembly, a second driving assembly and a clip cartridge assembly that are provided in the working head. The clip cartridge assembly comprises a clip cartridge configured to accommodate a ligation clip and a clip jaw provided on a side of the clip cartridge; the working head further comprises a firing rod and a clip-pushing rod, the firing rod is connected to the clip jaw to drive the clip jaw to open or close for firing the ligation clip, the first driving assembly is respectively connected to the firing rod and the first motor, the second driving assembly is respectively connected to the clip-pushing rod and the second motor, and the clip-pushing rod is configured to push the ligation clip in the clip cartridge to enter into the clip jaw.

For example, the first driving assembly comprises a first driving gear, a first nut and a first threaded rod, the first driving gear is provided on the first motor, gears engaging with each other are respectively provided on the first driving gear and the first nut, the first threaded rod is connected to the first nut by thread connection manner, and the first threaded rod is connected to the firing rod.

For example, the second driving assembly comprises a second driving gear, a second nut and a second threaded rod, the second driving gear is provided on the second motor, gears engaging with each other are respectively provided on the second driving gear and the second nut, the second threaded rod is connected to the second nut by thread connection manner, and the second threaded rod is connected to the clip-pushing rod.

For example, an end of the clip cartridge is provided with a mounting part, the clip jaw is mounted to the mounting part, a mounting hole is respectively on the clip jaw and the mounting part, and a mounting pin is inserted into the mounting holes of the clip jaw and the mounting part so that the clip jaw is mounted to the mounting part.

For example, the clip jaw comprises a first clip jaw sheet and a second clip jaw sheet, a connecting groove is respectively provided on the first clip jaw sheet and the second clip jaw sheet, the connecting groove provided on the first clip jaw sheet and the connecting groove provided on the second clip jaw sheet cross with each other, a connecting pin is provided on an end, close to the clip jaw, of the firing rod, and the connecting pin is provided in the connecting grooves of the first clip jaw sheet and the second clip jaw sheet.

For example, a clip delivery assembly configured for pushing the ligation clip to move in the clip cartridge is provided in the clip cartridge, a first channel and a second channel are provided on the clip cartridge, the clip-pushing rod and the clip delivery assembly are provided in the first channel, and the firing rod is provided in the second channel.

For example, the clip delivery assembly comprises a clip pushing block, the clip-pushing rod is connected to the clip pushing block, and the second driving assembly controls a position of the clip pushing block in the clip cartridge by driving the clip-pushing rod to move.

For example, an outer sleeve is sleeved on the clip cartridge, and a cutout for opening or firing of the clip jaw is provided at an end, close to the clip jaw, of the outer sleeve.

For example, the working head further comprises a support sleeve, the support sleeve is connected to the outer sleeve, the first channel and the second channel are further provided in the support sleeve, and the firing rod and the clip-pushing rod are provided in each of the support sleeve and the clip cartridge.

For example, the dual-motor continuous-firing clip applicator further comprises a handle, a control circuit is provided in the handle, and the control circuit is electrically connected to the first motor and the second motor.

Compared with the prior art, the beneficial effects of the embodiments of the present disclosure at least are as follows. The dual-motor clip applicator provided by the embodiments of the present disclosure uses the first motor and the second motor to respectively control the firing of the clip jaw and the clip delivery of the clip cartridge, such that the operation is simpler, the freedom degree of the operation is higher, the error rate is reduced, and the problems that the conventional clip applicator needs to complete the firing of the ligation clip and the delivery of the ligation clip by one shaft and thus has strong dependent and restrictive relationships between the firing of the ligation clip and the delivery of the ligation clip to easily cause operational errors and the like are solved.

Furthermore, according to the dual-motor clip applicator, the working head is separated into the support sleeve and the clip cartridge assembly that are connected to each other, the clip delivery assembly is provided in the clip cartridge, and the firing rod and the clip-pushing rod are provided in the support sleeve and the clip cartridge; and thus, the support sleeve is capable of being reused, so that clip cartridge assemblies of different specifications are mounted to meet the surgical requirements, thereby greatly reducing the cost and reducing the number of surgical instruments.

The above description is only an outline of the technical solutions of the embodiments of the present disclosure. In order to better understand the technical solutions of the embodiments of the present disclosure so that they can be implemented according to the contents of the specification, the following detailed description will be made with the exemplary embodiments of the present disclosure in conjunction with the accompanying drawings.

### BRIEF DESCRIPTION OF THE DRAWINGS

FIG. 1 is an external structural schematic diagram of a dual-motor clip applicator according to embodiments of the present disclosure.

FIG. 2 is an internal structural schematic diagram of the dual-motor clip applicator according to the embodiments of the present disclosure.

FIG. 3 is a structural schematic diagram of motors and drive assemblies of the dual-motor clip applicator according to the embodiments of the present disclosure.

FIG. 4 is a structural schematic diagram of a clip cartridge and a clip jaw of the dual-motor clip applicator according to the embodiments of the present disclosure.

FIG. 5 is a structural schematic diagram of a support sleeve of the dual-motor clip applicator according to the embodiments of the present disclosure.

### DETAILED DESCRIPTION

The exemplary specific modes of the present disclosure will be described in further detail below in conjunction with the drawings and embodiments. The following embodiments are intended to illustrate the present disclosure, but not to limit the scope of the present disclosure.

It should be noted that the terms such as “upper”, “lower”, “left”, “right”, “inner”, “outer” and the like in the present disclosure are only intended to describe the embodiments of the present disclosure by referring to the drawings, and are not used as limiting terms.

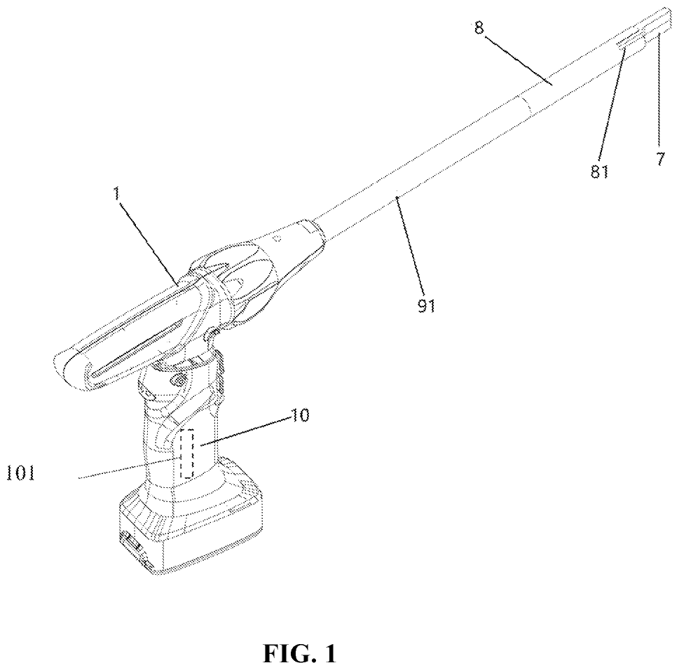

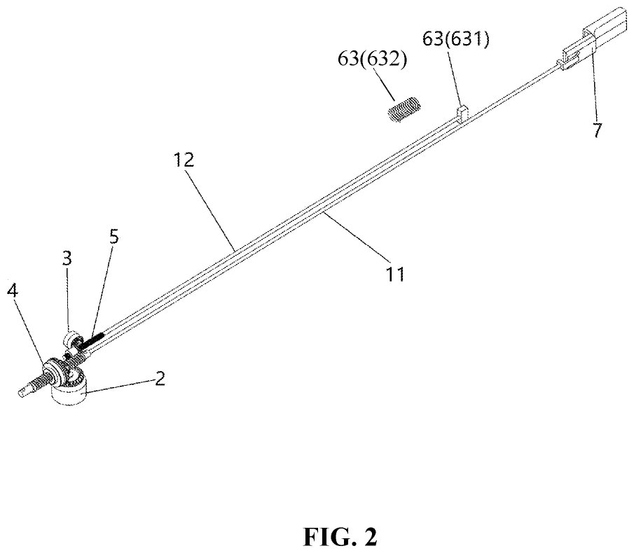

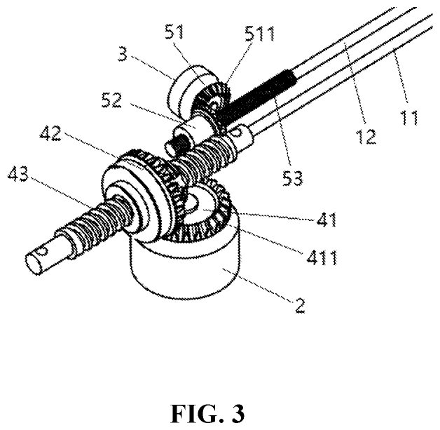

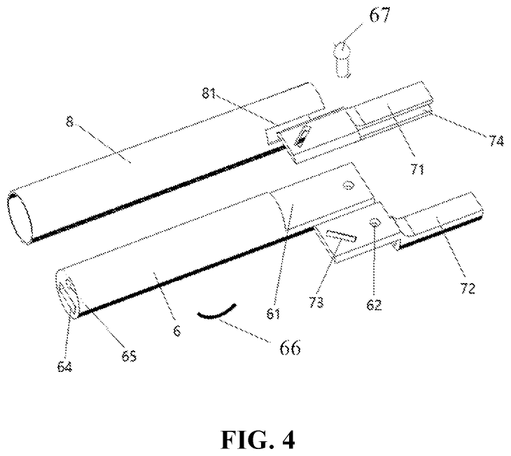

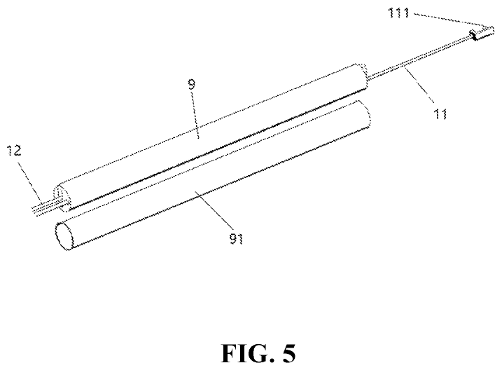

Referring to FIG. 1 to FIG. 5, a dual-motor clip applicator is provided by embodiments of the present disclosure, the dual-motor clip applicator includes a working head 1, and a first motor 2, a second motor 3, a first driving assembly 4, a second driving assembly 5 and a clip cartridge assembly that are provided in the working head 1; the clip cartridge assembly includes a clip cartridge 6 configured to accommodate a ligation clip 66 and a clip jaw 7 provided on a side of the clip cartridge 6; a firing rod 11 and a clip-pushing rod 12 are further provided in the working head 1, the firing rod 11 is connected to the clip jaw 7 to drive the clip jaw 7 to open or close for firing the ligation clip 66, the first driving assembly 4 is respectively connected to the firing rod 11 and the first motor 2, the second driving assembly 5 is respectively connected to the clip-pushing rod 12 and the second motor 3, and the clip-pushing rod 12 is configured to push the ligation clip 66 in the clip cartridge 6 to enter into the clip jaw 7.

In the embodiments of the present disclosure, the first driving assembly 4 includes a first driving gear 41, a first nut 42 and a first threaded rod 43, the first driving gear 41 is provided on the first motor 2, gears 411 engaging with each other are respectively provided on the first driving gear 41 and the first nut 42, the first threaded rod 43 is connected to the first nut 32 by thread connection manner, and the first threaded rod 43 is connected to the firing rod 11. The second driving assembly 5 includes a second driving gear 51, a second nut 52 and a second threaded rod 53, the second driving gear 51 is provided on the second motor 3, gears 511 engaging with each other are respectively provided on the second driving gear 51 and the second nut 52, the second threaded rod 53 is connected to the second nut 52 by thread connection manner, and the second threaded rod 53 is connected to the clip-pushing rod 12.

In the embodiments of the present disclosure, an end of the clip cartridge 6 is provided with a mounting part 61, and the clip jaw 7 is mounted to the mounting part 61. For example, a mounting hole 62 is respectively provided on the clip jaw 7 and the mounting part 61; and a mounting pin 67 is inserted into the mounting holes 62 so that the clip jaw 7 is mounted to the mounting part 61. The clip jaw 7 includes a first clip jaw sheet 71 and a second clip jaw sheet 72, a connecting groove 73 is respectively provided on the first clip jaw sheet 71 and the second clip jaw sheet 72, and the connecting groove 73 provided on the first clip jaw sheet 71 and the connecting groove 73 provided on the second clip jaw sheet 72 cross with each other. A positioning groove 74 is provided on the first clip jaw sheet 71 and the second clip jaw sheet 72. After entering the clip jaw 7, the ligation clip is accommodated in the positioning grooves 74. A connecting pin 111 is provided on an end, close to the clip jaw 7, of the firing rod 11, and the connecting pin 111 is fixedly provided in the connecting grooves 73, such that the firing rod 11 drives the clip jaw 7 to open or close for firing the ligation clip. In the embodiments, an X-shaped arrangement is used. The clip jaw 7 is mounted on the mounting part 61; if the firing rod 11 pushes the clip jaw 7 outwards, the connecting pin 111 provides an outward push force for the first clip jaw sheet 71 and the second clip jaw sheet 72, such that the clip jaw 7 opens; and if the firing rod 11 pulls the clip jaw 7 inwards, the connecting pin 111 provides an inward pull force for the first clip jaw sheet 71 and the second clip jaw sheet 72, such that the clip jaw 7 closes and fires.

For example, a clip delivery assembly 63 configured for pushing the ligation clip to move in the clip cartridge 6 is provided in the clip cartridge 6. The clip delivery assembly 63 includes a clip pushing block 631. The clip pushing block 631 abuts against and pushes the ligation clip, and the clip-pushing rod 12 is connected to the clip pushing block 631. In the embodiments, the clip delivery assembly 63 for example further includes a spring 632. The spring 632 is connected with the clip pushing block 631 and plays a reset role. A first channel 64 and a second channel 65 are provided on the clip cartridge 6. The clip-pushing rod 12 and the clip delivery assembly 63 are provided in the first channel 64, and the firing rod 11 is provided in the second channel 65. The second driving assembly 5 controls a position of the clip pushing block 631 in the clip cartridge 6 by driving the clip-pushing rod 12 to move, so as to drive the ligation clip to move. With the above arrangement, the electric and continuous firing of the ligation clips in the clip cartridge 6 is implemented; and theoretically, there are no limits made on the total number of the ligation clips in the clip cartridge 6.

In the embodiments of the present disclosure, the clip cartridge assembly further includes an outer sleeve 8, the outer sleeve 8 is provided in the working head 5 and is sleeved on the clip cartridge 6, and a cutout 81 for the opening or firing of the clip jaw 7 is provided at an end, close to the clip jaw 7, of the outer sleeve 8. The working head 1 further includes a support sleeve 9 that is connected to the outer sleeve 8 and is also provided with the first channel 64 and the second channel 65, and the firing rod 11 and the clip-pushing rod 12 are respectively provided in the first channel 64 and the second channel 65 of each of the support sleeve 9 and the clip cartridge 6. With such a design, the support sleeve 9 is used repeatedly without replacement, but the clip cartridges 6 of different models and specifications are mounted for surgical operation. In the embodiments, a protective sleeve 91 is sleeved on the support sleeve 9, and the protective sleeve 91 is connected to the outer sleeve 8.

The dual-motor continuous-firing clip applicator provided by the embodiments of the present disclosure further includes a handle 10, a control circuit 101 is provided in the handle 10, the control circuit 101 is electrically connected to the first motor 2 and the second motor 3, and the control circuit 101 comprises a motor control module, a support sleeve identification module, a clip delivery and clip firing control module, a battery detection and control module, a working status indicator module and a data record storage module that are controlled by single-chip processor.

In the control circuit 101:

The motor control module is configured for controlling the switching-on and switching-off of a drive motor; a closing/opening button (not shown) mounted at a lower portion of the handle 10 is isolated from an inner part of the main body of the clip applicator; and the motor control is realized by a magnet inside the button being close to or away from a Hall switch sealed and packaged in the handle.

The support sleeve identification module transmits the data about the type of the support sleeve by a terminal (not shown) provided on the support sleeve 8, and then the control circuit starts a corresponding working program based on the data.

The clip delivery and clip firing control module, together with a signal identification module (not shown), a photoelectric limit switch position control module, and a safety module that are mounted in the support sleeve 8, realizes the delivery of the ligation clip and the firing of the ligation clip.

The battery detection and control module provides a battery power detection function, and monitors the battery power before and during the surgical operation to ensure that the battery power used during the surgical operation meets the needs of the surgical operation or the battery is replaced in time.

The working status indicator module provides a status of the battery power, a support sleeve connection status, a status of completing the delivery of the ligation clip, and a status of completing the firing of the ligation chip by a display.

The dual-motor clip applicator provided by the embodiments of the present disclosure operates as follows. Initially, a plurality of ligation clips are provided in the clip cartridge 6, and the clip pushing block 631 connected to the clip-pushing rod 12 is provided at a side of the ligation clips away from the clip jaw 7; during the process of delivering the ligation clip into the clip jaw, by pressing the button on the handle 10, the firing rod 11 drives the clip jaw 7 to open, and at this time, the clip-pushing rod 12 is pushed forwards, thereby driving the clip pushing block 631 to push the ligation clip to enter the positioning groove 74 of the clip jaw 7; after the ligation clip reaches a position of a target vessel or tissue, by pressing the button on the handle 10, the firing rod 11 drives the clip jaw 7 to pull back and close such that the target vessel or tissue is ligated, thereby completing the firing. Then, the above procedures are repeated to complete the firing of the next ligation clip.

Compared with the prior art, the beneficial effects of the embodiments of the present disclosure at least are as follows. The dual-motor clip applicator provided by the embodiments of the present disclosure uses the first motor and the second motor to respectively control the firing of the clip jaw and the clip delivery of the clip cartridge, such that the operation is simpler, the freedom degree of the operation is higher, the error rate is reduced, and the problems that the conventional clip applicator needs to complete the firing of the ligation clip and the delivery of the ligation clip by one shaft and thus has strong dependent and restrictive relationships between the firing of the ligation clip and the delivery of the ligation clip to easily cause operational errors and the like are solved.

Furthermore, according to the dual-motor clip applicator, the working head is separated into the support sleeve and the clip cartridge assembly that are connected to each other, the clip delivery assembly is provided in the clip cartridge, and the firing rod and the clip-pushing rod are provided in the support sleeve and the clip cartridge; and thus, the support sleeve is capable of being reused, so that clip cartridge assemblies of different specifications are mounted to meet the surgical requirements, thereby greatly reducing the cost and reducing the number of surgical instruments.

In addition, the clip applicator of the embodiments of the present disclosure works automatically, is electrically driven and does not rely on the experience and status of a doctor; therefore, the stability of a ligation effect is good, and the doctor does not need to fire manually, which saves the doctor's physical strength, time and effort.

The technical features of the foregoing embodiments may be combined arbitrarily. In order to make the description concise, all possible combinations of the various technical features in the foregoing embodiments are not described. However, as long as there is no conflict in the combination of these technical features, all combinations should be considered as within the scope of the disclosure.

The foregoing embodiments merely are some of the embodiments of the present disclosure and the descriptions of the foregoing embodiments are specific and detailed, but the foregoing embodiments should not be understood as limiting of the scope of the disclosure. It should be pointed out that a person of ordinary skill in the art may make modifications and improvements without departing from the concept of the present disclosure, and all of these modifications and improvements belong to the protection scope of the present disclosure. Therefore, the protection scope of the present disclosure should be defined by the appended claims.

1. A dual-motor clip applicator, comprising a working head, and a first motor, a second motor, a first driving assembly, a second driving assembly and a clip cartridge assembly that are provided in the working head, wherein
the clip cartridge assembly comprises a clip cartridge configured to accommodate a ligation clip and a clip jaw provided on a side of the clip cartridge;
the working head further comprises a firing rod and a clip-pushing rod, the firing rod is connected to the clip jaw to drive the clip jaw to open or close for firing the ligation clip, the first driving assembly is respectively connected to the firing rod and the first motor, the second driving assembly is respectively connected to the clip-pushing rod and the second motor, and the clip-pushing rod is configured to push the ligation clip in the clip cartridge to enter into the clip jaw.

2. The dual-motor clip applicator according to claim 1, wherein the first driving assembly comprises a first driving gear, a first nut and a first threaded rod, the first driving gear is provided on the first motor, gears engaging with each other are respectively provided on the first driving gear and the first nut, the first threaded rod is connected to the first nut by thread connection manner, and the first threaded rod is connected to the firing rod.

3. The dual-motor clip applicator according to claim 1, wherein the second driving assembly comprises a second driving gear, a second nut and a second threaded rod, the second driving gear is provided on the second motor, gears engaging with each other are respectively provided on the second driving gear and the second nut, the second threaded rod is connected to the second nut by thread connection manner, and the second threaded rod is connected to the clip-pushing rod.

4. The dual-motor clip applicator according to claim 1, wherein an end of the clip cartridge is provided with a mounting part, the clip jaw is mounted to the mounting part.

5. The dual-motor clip applicator according to claim 1, wherein the clip jaw comprises a first clip jaw sheet and a second clip jaw sheet, a connecting groove is respectively provided on the first clip jaw sheet and the second clip jaw sheet, the connecting groove provided on the first clip jaw sheet and the connecting groove provided on the second clip jaw sheet cross with each other, a connecting pin is provided on an end, close to the clip jaw, of the firing rod, and the connecting pin is provided in the connecting grooves of the first clip jaw sheet and the second clip jaw sheet.

6. The dual-motor clip applicator according to claim 1, wherein a clip delivery assembly configured for pushing the ligation clip to move in the clip cartridge is provided in the clip cartridge, a first channel and a second channel are provided on the clip cartridge, the clip-pushing rod and the clip delivery assembly are provided in the first channel, and the firing rod is provided in the second channel.

7. The dual-motor clip applicator according to claim 6, wherein the clip delivery assembly comprises a clip pushing block, the clip pushing block abuts against and pushes the ligation clip, the clip-pushing rod is connected to the clip pushing block, and the second driving assembly controls a position of the clip pushing block in the clip cartridge by driving the clip-pushing rod to move, so as to drive the ligation clip to move.

8. The dual-motor clip applicator according to claim 6, wherein an outer sleeve is sleeved on the clip cartridge, and a cutout for opening or firing of the clip jaw is provided at an end, close to the clip jaw, of the outer sleeve.

9. The dual-motor clip applicator according to claim 8, wherein the working head further comprises a support sleeve, the support sleeve is connected to the outer sleeve, the first channel and the second channel are further provided in the support sleeve, and the firing rod and the clip-pushing rod are respectively provided in the first channel and the second channel of each of the support sleeve and the clip cartridge.

10. The dual-motor clip applicator according to claim 1, wherein the dual-motor clip applicator further comprises a handle, a control circuit is provided in the handle, and the control circuit is electrically connected to the first motor and the second motor.

11. The dual-motor clip applicator according to claim 2, wherein the second driving assembly comprises a second driving gear, a second nut and a second threaded rod, the second driving gear is provided on the second motor, gears engaging with each other are respectively provided on the second driving gear and the second nut, the second threaded rod is connected to the second nut by thread connection manner, and the second threaded rod is connected to the clip-pushing rod.

12. The dual-motor clip applicator according to claim 4, wherein a mounting hole is respectively on the clip jaw and the mounting part, and a mounting pin is inserted into the mounting holes of the clip jaw and the mounting part so that the clip jaw is mounted to the mounting part.

13. The dual-motor clip applicator according to claim 5, wherein a positioning groove is provided on the first clip jaw sheet and the second clip jaw sheet; and after entering into the clip jaw, the ligation clip is accommodated in the positioning grooves.

14. The dual-motor clip applicator according to claim 7, wherein the clip delivery assembly further includes a spring, and the spring is connected with the clip pushing block and plays a reset role.

15. The dual-motor clip applicator according to claim 9, wherein a protective sleeve is sleeved on the support sleeve, and the protective sleeve is connected to the outer sleeve.
